(19) United States
(12) Patent Application Publication
(10) Pub. No.: US 2002/0150876 A1
(43) Pub. Date: Oct. 17, 2002

(54) Selectable marker genes

(76) Inventors: Susanne Dagmar Pippig, San Francisco, CA (US); Gabor Veres, Palo Alto, CA (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/817,487

(22) Filed: Mar. 26, 2001

Related U.S. Application Data
(60) Provisional application No. 60/266,331, filed on Mar. 30, 2000.

(51) Int. Cl. C12Q 1/00; C12Q 1/68; C12N 15/867; G01N 33/567

(52) U.S. Cl. 435/4; 435/6; 435/456; 435/7.2

(57) ABSTRACT

The present invention concerns a method of using a muscle specific tyrosine kinase receptor molecule (MuSK-R) or a mutated MuSK-R (mMuSK-R) thereof as a selectable marker in mammalian cells, particularly human cells. Preferred markers are mMuSK-Rs incapable of signal transduction and preferably molecules wherein the intracellular domain has been modified by deletion of the signaling region. The invention also relates to a method of identifying genetically modified mammalian cells including introducing a mMuSK-R into a target cell as a selectable marker. Further a method for the immunoselection of transduced mammalian cells is disclosed comprising identifying the transduced cells by incubation of the cells with an antibody which recognizes and binds specifically to a MuSK-R or mMuSK-R thereof.

This application claims the benefit under 35 USC §119(e) of the following United States provisional patent application: Provisional Application serial number to be assigned, filed Mar. 30, 2000, as U.S. application Ser. No. 09/539,248, for “Selectable Marker Genes,” and subject to a Petition for Conversion to Provisional Application, filed Nov. 16, 2000.

### BACKGROUND OF THE INVENTION

This invention relates to a method of identifying genetically modified mammalian cells, particularly human cells using a muscle specific tyrosine kinase receptor molecule (MuSK-R) or a mutated MuSK (mMuSK-R) thereof as a selectable cell marker.

The use of selectable markers is well known for the identification of prokaryotic and eukaryotic cells, and the use of these markers is essential because frequently when a DNA sequence of interest is introduced into a cell it will not necessarily lead to a phenotype that is readily determined. The number of selectable markers used in identifying eukaryotic cells and especially mammalian cells has been limited. In the past, selectable markers that conferred drug resistance have been employed (i.e. G-418 and hygromycin). More recently, selectable markers that are combined with fluorescence activated cell sorting (FACS) have been used, for example, green fluorescent protein (GFP). Alternatively, antibodies that recognize a cell surface molecule may be coupled to a fluorophore to help identify the cells of interest.

Several cell surface molecules have been used as selectable cell markers including murine CD8, CD24, and human Low-Affinity Nerve Growth Factor Receptor (NGFR). Reference is made to the following publications; WO95/06723; WO98/19540; Jolly et al., Proc. Natl. Acad. Aca. 80:477 (1983); Reddy et al., Mol. Brain Res. 8:137 (1990); and Valenzuela et al., Neuron 15:573 (1995). Cell surface selectable markers offer an advantage over drug resistance cell markers in that identification and selection of the genetically modified cells may be performed in a shorter time frame. Additionally, if a selectable marker is a human protein it may prevent an immune reaction in a human treated with cells expressing the selectable marker. Therefore, it is an object of the present invention to provide a method of identifying genetically modified mammalian cells with a cell surface receptor molecule wherein the cell surface receptor would function as a selectable marker, would have a restricted expression pattern, would not be active in the target cell, and could be identified and selected with anti-marker antibodies. This object has been accomplished by a method of identifying genetically modified cells expressing a MuSK-R or a mMuSK-R.

### SUMMARY OF THE INVENTION

Accordingly the invention provides for a method of identifying genetically modified mammalian cells comprising introducing a nucleic acid sequence encoding a mutated muscle specific tyrosine kinase receptor (mMuSK-R) operatively linked to a promoter into a mammalian cell to form a genetically modified cell; allowing expression of the mMuSK-R in the genetically modified cell; and identifying the cells expressing the mutant MuSK-R. In one embodiment the mMuSK-R is a MuSK-R sequence having at least 150 amino acids deleted from the intracellular domain. In a second embodiment the mMuSK-R is a MuSK-R sequence having the kinase catalytic site deleted. In a third embodiment a leader sequence is added to the mMuSK-R. A preferred mMuSK-R is derived from the hMuSK-R sequence illustrated in SEQ ID NO. 1 and SEQ ID NO. 2. Preferably the mMuSK-R is mMuSK-RI or mMuSK-RII. In a further embodiment the identifying step is accomplished by contacting the genetically modified cells with an antibody. In another embodiment the nucleic acid sequence encoding the mMuSK-R is introduced into the mammalian cells by a vector, preferably a retroviral vector. Hematopoietic cells are the preferred target cells, particularly hematopoietic stem cells and T-cells.

In another aspect, the invention provides a vector comprising a nucleic acid sequence encoding a mMuSK-R operatively linked to a promoter wherein the mMuSK-R is derived from the sequence ser forth in SEQ ID NO. 1 or a sequence substantially similar to said sequence. Preferably the mMuSK-R is the molecule designated mMuSK-RI or mMuSK-RII.

In a third aspect, the invention includes a method of identifying genetically modified human hematopoietic cells comprising introducing a nucleic acid sequence encoding a muscle specific tyrosine kinase receptor (MuSK-R) into a human hematopoietic cell; allowing expression of the MuSK-R in said cells; and identifying the genetically modified hematopoietic cells from the non-modified hematopoietic cells.

In yet a further aspect, the invention provides a method of identifying genetically modified human hematopoietic cells comprising incorporating a nucleic acid sequence encoding a mMuSK-R into a population of human hematopoietic cells; introducing a heterologous DNA sequence which encodes a protein of interest into the population of human hematopoietic cells; allowing expression of the mMuSK-R in said cells; and identifying the genetically modified cells expressing the mMuSK-R. Preferably the heterologous DNA sequence encoding the protein of interest and the nucleic acid sequence encoding the mMuSK-R are introduced into the cells on the same vector, preferably a retroviral vector.

A further aspect of the invention pertains to a method for the immunoselection of transduced mammalian cells comprising transducing cells with a nucleic acid sequence encoding a mMuSK-R; incubating the cells with an antibody which recognizes and binds specifically to the mMuSK-R; and identifying the bound transduced cells.

Another aspect of the invention includes a method of identifying mammalian cells expressing a protein of interest, comprising introducing into a population of mammalian cells a nucleic acid sequence encoding a mMuSK-R, wherein said mMuSK-R can not effect signal transduction; introducing a heterologous DNA sequence encoding a protein of interest into said population; culturing the mammalian cells under conditions which favor growth and expansion of said cells; and identifying cells which express the mMuSK-R thereby obtaining cells which express the protein of interest.

Another aspect of the invention pertains to a method of identifying mammalian cells comprising introducing a nucleic acid sequence encoding a mutated muscle specific tyrosine kinase receptor (mMuSK-R) operatively linked to a promoter into a mammalian cell to form a genetically modified cell; allowing expression of the mMuSK-R; exposing the cells to a monoclonal antibody wherein said antibody recognizes and binds to the cells expressing the mMuSK-R and does not bind to the cells lacking expression of mMuSK-R; and separating the cells that bind to the monoclonal antibody from cells that do not bind to the antibody.

### BRIEF DESCRIPTION OF THE DRAWINGS

FIG. 1 is a schematic representation of a wild type MuSK-R and a mMuSK-R wherein the cytoplasmic domain has been truncated. A leader sequence has been added to the 5′ end of the sequence as a tag.

FIG. 2 illustrates a MuSK-R designated hMuSK-R and corresponds to the nucleic acid sequence as set forth in SEQ ID NO: 1 and the amino acid sequence as set forth in SEQ ID NO: 2. The signal peptide includes amino acid residues 1-19. The extracellular domain is represented by amino acid residues 20-493. The transmembrane domain includes amino acid residues 494-515, and the cytoplasmic domain includes amino acid residues 516-869.

FIG. 3 is a schematic representation of the pSeqTag2bhMuSK-R.

FIG. 4 illustrates the expression of MuSK-R on CEMSS cells and CEMSS MuSK-R cells using the monoclonal antibodies H1 (B.), H2 (C.) and H4 (D.).

FIG. 5 illustrates expression of hMuSK-R (SEQ ID NO: 1) in nontransduced CEMSS cells (A.) and the expression of hMuSK-R (B.) and mMuSK-RII (D.) on CEMSS cells transduced with PPA-6 supernatants that express hMuSK-R or mMuSK-RII respectively. Both popuations were enriched after immuno-magnetic bead selection using monoclonal antibody H2 as illustrated for hMuSK-R (C.) and mMuSK-RII (E.).

### DETAILED DESCRIPTION OF THE INVENTION

The practice of the present invention will employ, unless otherwise indicated conventional techniques of cell biology, molecular biology, cell culture, immunology and the like, which are in the skill of one in the art. These techniques are fully disclosed in the current literature and reference is made specifically to Sambrook, Fritsch and Maniatis eds., “Molecular Cloning A Laboratory Manual, 2^{nd} Ed., Cold Springs Harbor Laboratory Press, (1989); Celis, J. E. “Cell Biology, A Laboratory Handbook”, Academic Press Inc., (1994); Coligan et al., “Current Protocols in Immunology”, John Wiley & Sons (1991); Harlow et al., “Antibodies: A Laboratory Manual”, Biosupplynet Source Book, Cold Springs Harbor Laboratory Press (1999); and Horton, R. M. “Methods in Molecular Biology” Vol. 15: PCR Protocols (1993).

All publications and patent applications cited in the specification are indicative of the level of skill of those in the art to which this invention pertains, and are hereby incorporated by reference in their entirety.

As used in this specification and the claims, the singular form “a”, “an” and “the” include plural references unless the context clearly dictates otherwise. For example, a stem cell includes a plurality of stem cells.

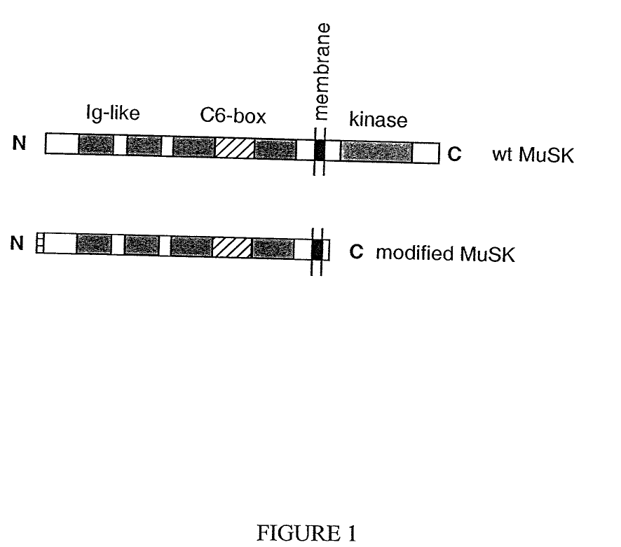

The selectable marker of the instant invention is a muscle specific tyrosine kinase receptor molecule (MuSK-R) or a mutation thereof (mMuSK-R). MuSK-R is believed to initiate the formation of neuromuscular junctions in response to agrin (Glass, et al. Cell 85:513 (1996)). The domain structure of a MuSK-R is schematically illustrated in FIG. 1. MuSK-R is comprised of a signal sequence or leader sequence that targets the protein to the secretory pathway. The extracellular domain follows the signal sequence. This domain is made up of several hundred amino acids, and while the exact number of amino acid residues vary, typically the extracellular domain includes around 500 amino acids. The extracellular domain is the part of the receptor that normally projects from the cell into the extracellular environment and includes a ligand binding region. The extracellular domain is one of the most distinctive features of the kinase receptors. In MuSK-R, the extracellular domain contains immunoglobulin-like (Ig-like) regions. Typically four Ig-like regions are found. However there are reports of MuSK-Rs with three Ig-like regions. The extracellular domain may include 6 contiguous cysteine residues known as a C6-box. While the location of the C6-box may vary depending on the particular MuSK-R, in certain MuSK-Rs it is found approximately at amino acid residues 373-382. The transmembrane domain is generally localized in the cell membrane and consists of a stretch of hydrophobic residues followed by several basic residues. The intracellular domain (used interchangeably with the cytoplasmic domain) includes the catalytic part of themolecule and is positioned within the cell.

MuSK-Rs are also known in the art as denervated muscle kinase receptors and have been referred to as DmKs (see U.S. Pat. No. 5,656,473 and particularly SEQ ID NOS: 16 and 17 therein). MuSK-R sequences have been isolated and identified from humans, rats, mice, and xenopus. Closely related to human MuSK-R is a receptor isolated from the electric ray Torpedo californica designated Torpedo tyrosine kinase receptor, and ROR tyrosine kinase receptors (Jennings, et al. Proc. Natl. Acad. Sci. USA 90:2895 (1993); Masiakowski et al., J. Biol. Chem. 267: 26181-26190 (1992); Valenzuela et al., Neuron, 15:573-584 (1995); and Hesser et al., FEBS Letters, 442:133-137 (1999)).

Other non-limiting examples of MuSK-Rs available from public depositories such as GeneBank and ATCC include accession numbers: NM005592; AF006464; A448972; AI800924; AI700028; AI341265; AI341122; AI302067; U34985; AA448972; and ATCC 75498. As mentioned above MuSK-R is specific to the skeletal muscle lineage.

The term MuSK-R as used in the present specification and claims is broadly defined to include the known MuSK-Rs (including DmK receptors), isoforms or variants of known MuSK-Rs having similar structure, tyrosine kinase receptors that are functionally similar to known MuSK-Rs and novel MuSK-Rs not previously described that are identified using screening techniques well known to those in the art. Such techniques may include the use of degenerate oligodeoxyribonucleotide primers.

Accordingly, the term MuSK-R when referring to a nucleic acid molecule includes (a) nucleic acid sequences comprising a coding region of a known mammalian MuSK-R; (b) a nucleic acid sequence which hybridizes under stringent conditions to the nucleic acid of (a) and which encodes a mammalian MuSK-R; and (c) a degenerate MuSK-R wherein the MuSK-R has undergone changes in its nucleic acid sequence that does not significantly effect the properties of the MuSK-R protein encoded by the polynucleotide. These changes include ones that do not change the encoded amino acid sequence, ones that result in conservative substitutions of amino acid sequences, or ones that result in one or a few amino acid deletions or additions. Suitable substitutions are known by those skilled in the art. Amino acid residues that can be conservatively substituted for one another include but are not limited to, glycine/alanine; valine/isoleucine/leucine, asparagine/glutamine; asparatic acid/glutamic acid; serine/threonine; lysine/arginine; and phenylalanine/tyrosine. Any conservative amino acid substitution not significantly affecting the properties of a MuSK-R is encompassed by the term. MuSK-R includes not only naturally occurring MuSK-Rs but also may include genetically engineered MuSK-Rs.

As used herein “MuSK-R” or “mMuSK-R” refer to nucleic acid sequences or protein as appropriate from context. Polynucleotides or nucleic acids of the invention may be in the form of RNA or in the form of DNA, which DNA includes cDNA, genomic DNA or synthetic DNA.

The term MuSK-R when referring to a polypeptide encompasses known MuSK receptors, isoforms or variants of MuSK-Rs, and functionally equivalent receptors. A functionally equivalent receptor is a MuSK-R that can compete with a known MuSK-R for binding. More specifically, a functionally equivalent MuSK-R has at least 40%, preferably at least 60%, and more preferably at least 80% identical amino acids to the sequence set forth in SEQ ID NO:2 and can compete with the MuSK-R illustrated in SEQ ID NO: 2 for ligand or substrate binding.

According to the invention MuSK-R or mMuSK-Rs are used as selective markers to identify genetically modified cells. The marker is introduced on a nucleic acid construct into a target cell that normally does not express a MuSK-R. The term “introduced” is broadly used herein to include inserted, incorporated and the like. When the MuSK-R or mMuSK-Rs are used as selectable markers the molecule no longer possesses signaling activity. Signaling activity has be generally defined as triggering a response pathway in the cytosol to the nucleus which ultimately leads to activation of transcription. The lack of signaling activity may be due to a) use of a MuSK-R in tissue or cells other than muscle (Glass et al., Cell 85:513-523 (1996)) or b) use of a mMuSK-R

While modifications of MuSK-R may be known, the method of identifying genetically modified cells comprising using a MuSK-R or mMuSK-R as a selectable marker is not known.

As stated above, the localization of MuSK-R is in muscle tissue and MuSK-R serves as the functional agrin receptor. Agrin is a nerve-derived factor that can induce molecular reorganizations at the motor endplate. Therefore, MuSK-R may be used as a selective marker in tissue other than muscle.

In a preferred embodiment, the selectable marker of the invention is a mMuSK-R. The modifications to a MuSK-R encompassing mMuSK-Rs include truncations and/or deletions of MuSK-Rs. The mutation may occur in the extracellular domain and/or the intracellular domain by means well known in the art. The mutation causes the molecule to be devoid of signaling activity. Preferably the extracellular domain should still be capable of binding an antibody. In general the smallest peptide fragment of the extracellular domain capable of binding an antibody would be approximately 15 amino acid residues, more preferably at least 50 amino acid residues.

A preferred MuSK-R according to the invention is the sequence set forth in SEQ ID NOs: 1 and 2, designated herein as hMuSK-R. The extracellular domain is encoded by nucleotides 1 through 1479, the transmembrane domain is encoded by nucleotides 1480 through 1545, and the intracellular domain is encoded by nucleotides 1546 through 2607. Other preferred MuSK-Rs are molecules closely related to the sequences set forth in SEQ ID NOS: 1 and 2. Examples of closely related sequences are the sequences set forth in U.S. Pat. No. 5,656,473 particularly SEQ ID NOS: 16 and 17.

Mutants of MuSK-R (mMuSK-Rs) are known and reference is made to Apel et al., Neuron 18:623-635 (1997). In the present invention, preferred modifications to a MuSK-R include modifications to the cytoplasmic domain such as deletions of at least 150, preferably at least 200, more preferably at least 250, more preferably 300, and most preferably at least 350 amino acids of the cytoplasmic domain. The deletions are preferably truncations. Deletions or truncations may include deletion of tyrosine phosphorylation sites in the range of 1 to 19, preferably 2-15, more preferably 2-10 sites. Additionally the kinase catalytic site may be deleted from a MuSK-R. In one aspect, the kinase catalytic site is found at approximately amino acid residues 672 to 691 of SEQ ID NO.2. As long as the protein is stably expressed, there is no limitation to the number of sequences deleted or truncated in the cytoplasmic domain.

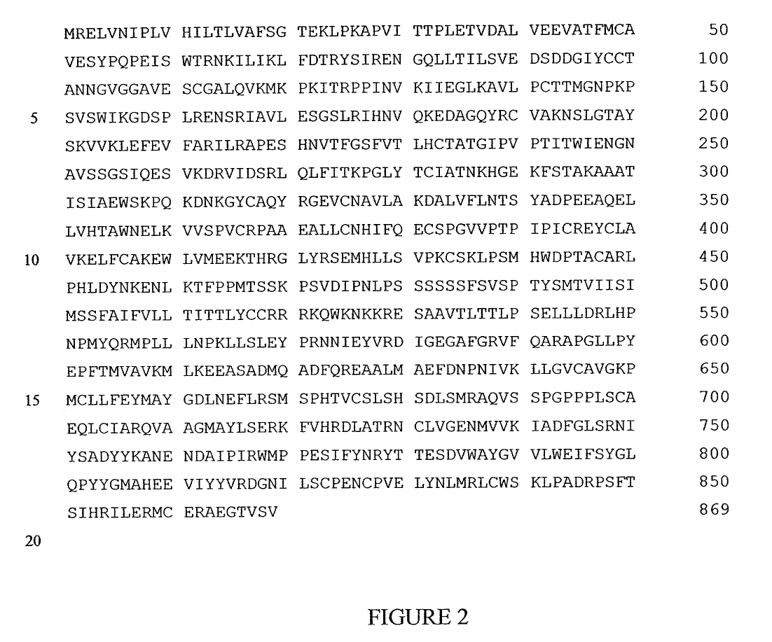

Particularly preferred mMuSK-Rs useful as selectable markers according to the invention include modifications to the MuSK-R sequence set forth in FIG. 2 (SEQ ID NO:2). In one embodiment the MuSK-R is truncated by least 300 amino acid residues in the cytoplasmic domain. One preferred embodiment includes the deletion of amino acid sequence 538-869 and is designated mMuSK-RI. Another preferred embodiment includes the deletion of amino acid sequence 577-869 and is designated mMuSK-RII.

In addition to modification of the cytoplasmic domain mutations may be made in the extracellular domain. The extracellular domain modification may include deletion of at least about 100 amino acids, preferably at least about 150 amino acids, more preferably at least about 200 amino acids, and still more preferably at least about 250 amino acids. A MuSK-R or mMuSK-R used as a selectable marker according to the invention preferably should contain an antibody-binding site in the extracellular domain.

General strategies for creating mutations in nucleic acids and proteins are well known. These methods may be used to create mMuSK-Rs from MuSK-R that are then useful as selectable markers for the identification of genetically modified cells. Both random and site-directed mutagenesis methods may be effective to create mutations in MuSK-Rs. Random methods encompass altering the sequences within restriction endonuclease sites, inserting an oligonucleotide linker randomly into a plasmid, using chemicals to damage plasmid DNA, and incorporating incorrect nucleotides during in vitro DNA synthesis. However, site-directed mutagenesis may be a more beneficial tool. Particularly preferred site-directed methods include oligonucleotide-directed mutagenesis and polymerase chain reaction (PCR) amplified oligonucleotide mutagenesis. These methods are known and reference is made to Wu et al., eds. Methods in Enzymology, Vol. 154: Recombinant DNA, Part E, Academic, NY (1987); Landt et al., Gene 96:125-129 (1990); Kirchhoff et al., Methods Mol. Biol. 57:323-333 (1995); and Sambrook et al., supra.

The usefulness of a MuSK-R or mMuSK-R as a selectable marker concerns the ability to select genetically modified cells in vitro, ex vivo and in vivo. While the MuSK-R or mMuSK-R may be introduced into a target cell as part of a nucleic acid construct operatively linked to a promoter, in a preferred embodiment the selectable marker of the invention is placed in a vector and then introduced into a target cell. As used herein “operatively linked” refers to an arrangement of elements wherein the components are configured so as to perform their usual function. To be operably linked a promoter or other control elements need not be contiguous with the coding sequence.

With respect to the nucleic acid constructs and vectors comprising the MuSK-R or mMuSK-R selectable markers, the choice of a promoter is well within the skill of one in the art and extends to any prokaryotic, eukaryotic or viral promoter capable of directing gene transcription in a target cell modified with a selectable marker of the invention. The promoter may be a tissue specific promoter, inducible promoter, synthetic promoter or hybrid promoter. More than one promoter may be used. Examples of promoters include but are not limited to; the phage lamda (PL) promoter; SV40 early promoter; adenovirus promoters, such as adenovirus major late promoter (Ad MLP); herpes simplex (HSV) promoter; a cytomegalovirus (CMV) promoter, such as human CMV immediate early promoter; a long terminal repeat (LTR) promoter, such as MoMLV LTR; the U3 region promoter of the Moloney murine sarcoma virus; Granzyme A promoter; regulatory sequences of the metallothionin gene; CD34 promoter; CD8 promoter; thymidine kinase (TK) promoters, B19 parvovirus promoters; PGK promoter; and rous sarcoma virus (RSV) promoter. Additionally promoter elements from yeast and other fungi may be used such as Gal 4 promoter and the alcohol dehyrodenase (ADH) promoter. These promoters are available commercially from various sources such as Stratagene (La Jolla, Calif.). It is to be understood that the scope of the present invention is not to be limited to a specific promoter. Preferred promoters include LTR promoters such as the 5′ LTR promoter of MoMLV, MSCV and HIV, and CMV promoters. In addition to promoters, other expression control sequences may be incorporated into the nucleic acid constructs used for identifying genetically modified cells according to the invention. Some of these regulatory sequences are enhancers, polyadenylation signals, RNA polymerase binding sequences, sequences conferring inducibility of transcription and other expression control elements such as scaffold attachment regions (SARs).

Vectors containing both a promoter and a cloning site into which a polynucleotide sequence can be operatively linked are well known in the art. Such vectors are capable of transcribing RNA in vitro or in vivo, and are commercially available from sources such as Stratagene (La Jolla, Calif.) and Promega Biotech (Madison, Wis.). Examples of vectors include vectors derived from viruses, such as baculovirus, retroviruses, adenoviruses, adeno-associated viruses, and herpes simplex viruses; bacteriophages; cosmids; plasmid vectors; fungal vectors; synthetic vectors; and other recombination vehicles typically used in the art. These vectors have been described for expression in a variety of eukaryotic and prokaryotic hosts and may be used for simple protein expression.

Specific examples of vectors include pSG, pSV2CAT, and pXt1 from Stratagene and pMSG, pSVL, pBPV and pSVK3 from Pharamacia. Other exemplary vectors include the pCMV mammalian expression vectors, such as pCMV6b and pCMV6c (Chiron Corporation), pSFFV-Neo, and pBluescript-SK+. In order to optimize expression and/or in vitro transcription, it may be necessary to remove, add or alter 5′ and/or3′ untranslated portions of polynucleotides to eliminate potentially extra inappropriate alternative translation initiation codons or other sequences that may interfere with or reduce expression, either at the level of transcription or translation. Alternatively consensus ribosome binding sites can be inserted 5′ or 3′ to the selective marker to enhance expression.

Particularly preferred vectors are retroviral vectors and reference is made to Coffin et al., “Retroviruses”, (1997) Chapter 9 pp; 437-473 Cold Springs Harbor Laboratory Press. Retroviral vectors useful in the invention are produced recombinantly by procedures already taught in the art. WO94/29438, WO97/21824 and WO97/21825 describe the construction of retroviral packaging plasmids and packing cell lines. Common retroviral vectors are those derived from murine, avian or primate retroviruses. The most common retroviral vectors are those based on the Moloney murine leukemia virus (MoMLV) and mouse stem cell virus (MSCV). Vectors derived from MoMLV include, Lmily, LINGFER, MINGFR, MND and MINT (Bender et al., J. Virol. 61: 1639-1649 (1987); Miller et al., Biotechniques 7: 980-990 (1989); Robbin et al. J. Virol., 71:9466-9474 (1997); and U.S. Pat. No. 5,707,865). Vectors derived from MSCV include MSCV-MiLy (Agarwal et al., J. of Virology 72:3720). Further non-limiting examples of vectors include those based on Gibbon ape leukemia virus (GALV), Moloney murine sacroma virus (MoMSV), myeloproliferative sarcoma virus (MPSV); murine embryonic stem cell virus (MESV), spleen focus forming virus (SFFV), and the lentiviruses, such as human immunodeficiency virus (HIV-1 and HIV-2). New vector systems are continually being developed to take advantage of particular properties of parent retroviruses such as host range, usage of alternative cell surface receptors and the like (C. Baum et al, Chap 4 in Gene Therapy of Cancer Cells eds. Lattime and Gerson (1998)). The present invention is not limited to particular retroviral vectors, but may include any retroviral vector. Particularly preferred vectors include DNA from a murine virus corresponding to two long terminal repeats, and a packaging signal. In one embodiment the vector is a MoMLV or MSCV derived vector and particularly MND (U.S. Pat. No. 5,707,865 and Norris et al., J. Virol. Methods, 75:161-167 (1998)).

In producing retroviral vector constructs, the viral gag, pol and env sequence will generally be removed from the virus, creating room for insertion of foreign DNA sequences. Genes encoded by foreign DNA are usually expressed under the control a strong viral promoter in the long terminal repeat (LTR). While the LTR promoter is preferred, as mentioned above numerous promoters are known.

Non-limiting preferred vector constructs according to the present invention include the general structure as outlined below in the 5′ to 3′ direction:

(a) LTR-X-I-mMuSKR-LTR;

(b) LTR-mMuSKR-LTR;

(c) LTR-mMuSKR-(I)-LTR;

(d) LTR-X-pmMuSKR-LTR;

(e) LTR-X-I-mMuSKR-SAR-LTR; and

(f) CMV-X-pmMuSKR-LTR.

wherein LTR is a long terminal repeat, X is a heterologous gene for a desired protein, mMuSKR is a selectable marker, p is a second promoter; I is an internal ribosomal binding site, SAR is a scaffold attachment region, and CMV is a cytomegalovirus promoter.

Such a construct can be packaged into viral particles efficiently if the gag, pol and env functions are provided in trans by a packaging cell line. Therefore when the vector construct is introduced into the packaging cell, the gag-pol and env proteins produced by the cell, assemble with the vector RNA to produce infectious virions that are secreted into the culture medium. The virus thus produced can infect and integrate into the DNA of the target cell, but does not produce infectious viral particles since it is lacking essential packaging sequences. Most of the packaging cell lines currently in use have been transfected with separate plasmids, each containing one of the necessary coding sequences, so that multiple recombination events are necessary before a replication competent virus can be produced. Alternatively the packaging cell line harbors a provirus. (The DNA form of the reverse-transcribed RNA once its integrates into the genomic DNA of the infected cell). The provirus has been crippled so that although it may produce all the proteins required to assemble infectious viruses, its own RNA can not be packaged into virus. RNA produced from the recombinant virus is packaged instead. Therefore, the virus stock released from the packaging cells contains only recombinant virus. Non-limiting examples of retroviral packaging lines include PA12, PA317, FLYA13, PE501, PG13, ΨCRIP, RD114, GP7C-tTA-G10, ProPak-A (PPA-6), and PT67. Reference is made to Miller et al., Mol. Cell Biol. 6:2895 (1986); Miller, et al., Biotechniques 7:980 (1989); Danos et al., Proc. Natl. Acad. Sci. USA 85:6460 (1988); Pear et al., Proc. Natl. Acad. Sci. USA 90:8392 (1993); Rigg et al., Virology 218:290 (1996); and Finer et al., Blood 83:43 (1994). Retroviral vector DNA can be introduced into packaging cells either by stable or transient transfection to produce vector particles.

Additionally preferred vectors include adenoviral vectors (Frey et al., Blood 91:2781 (1998) and WO95/27071) and adeno-associated viral vectors (Chatterjee et al., Current Topics in Microbiol. and Immunol. 218:61 (1996). Reference is also made to Shenk, Chapter 6, 161-178, Breakefield et al., Chapter 8 201-235; Kroner-Lux et al., Chapter 9, 235-256 in Stem Cell Biology and Gene Therapy, eds. Quesenberry et al., John Wiley & Sons, 1998 and U.S. Pat. Nos. 5,693,531 and 5,691,176. The use of adenovirus derived vectors may be advantageous under certain situations because they are capable of infecting non-dividing cells, and unlike retroviral DNA, the adenoviral DNA is not integrated into the genome of the target cell. Further the capacity to carry foreign DNA is much larger in adenoviral vectors than retroviral vectors. The adeno-associated viral vectors are another useful delivery system. The DNA of these viruses may be integrated into non-dividing cells, and a number of polynucleotides have been successfully introduced into different cell types using adeno-associated viral vectors. These vectors are capable of transducing several cell types including hematopoietic cells and epithelial cells.

In one embodiment, the construct or vector will include not only a nucleic acid sequence encoding a MuSK-R or mMuSK-R as a selective marker but also a second nucleic acid sequence encoding a protein of interest to be introduced into a target cell. In a preferred embodiment the nucleic acid molecules are DNA.

A protein of interest is broadly defined and includes for example, a therapeutic protein, a structural gene, a ribozyme, or an antisense sequence. The structural protein or gene may be the entire protein or only the functionally active fragment thereof. The protein may include for example one that regulates cell differentiation or a therapeutic gene capable of compensating for a deficiency in a patient that arises from a defective endogenous gene. Gene means a nucleic acid molecule the sequence which includes all the information required for the normal regulated production of a particular protein including the structural coding sequence. Additionally a therapeutic protein or gene may be one that antagonizes production or function of an infectious agent, antagonizes pathological processes, improves a host's genetic makeup, or facilitates engraftment.

Specific examples of a therapeutic gene or gene sequences are ones effective in the treatment of adenosine deaminase deficiency (ADA); sickle cell anemia; recombinase deficiency; recombinase regulatory gene deficiency; HIV such as an antisense or trans-dominant REV gene or a gene carrying a herpes simplex virus thymidine kinase (HSV-tk)). The second nucleic acid sequence may encode new antigens; drug resistant genes; a toxin; an apoptosis inducer effective to specifically kill cancerous cells; or a specific suicide gene. The therapeutic gene may be a non-human gene, for example a yeast gene (Seo et al., Proc. Natl. Acad. Sci. 95:9167 (1998)).

The vector or construct may also comprise, besides the second nucleic acid sequence encoding a protein of interest, a further DNA sequence. More than one gene may be necessary for the treatment of a particular disease. Alternatively more than one gene can be delivered using several compatible vectors. Depending on the genetic defect, the therapeutic gene can include regulatory and untranslated sequences. For human patients the therapeutic gene will generally be of human origin although genes of closely related species that exhibit high homology and biologically identical or equivalent function in humans may be used if the gene does not produce an adverse immune reaction in the recipient.

Nucleotide sequences for the protein of interest or a further DNA sequence will generally be known in the art or can be obtained from various sequence databases such as GeneBank. One skilled in the art will readily recognize that any structural gene can be excised as a compatible restriction fragment and placed in a vector in such a manner as to allow proper expression of the structural gene in target cells.

The target cells of the invention are mammalian cells that do not normally express ta MuSK-R. Mammalian cells include but are not limited to humans, mice, monkeys, farm animals, sport animals, pets, and other laboratory rodents and animals. Preferably the target cells are human cells. Preferred human cells include liver, hematopoietic, neural, endothelial vascular cells, tumor cells and epithelial cells. Hematopoietic cells are particularly preferred, and these cells encompass hematopoietic stem cells, erythrocytes, neutrophils, monocytes, platelets, mast cells, eosinophils and basophils, B and T lymphocytes and NK cells as well as the respective lineage progenitor cells. Hematopoietic stem cells and T-cells are especially preferred. Hematopoietic stem cells are defined as a population of hematopoietic cells containing long term mutlilineage repopulating potential. T-cells are defined as a type of lymphocyte and are thought to develop from hematopoietic stem cells.

Methods of obtaining target cells and particularly hematopoietic cells are known in the art and not repeated herein. Non-limiting sources of hematopoietic cells, including hematopoietic stem cells, are bone marrow, embryonic yolk sac, fetal liver tissue, adult spleen, and blood such as adult peripheral blood and umbilical cord blood. (To et al., Blood 89:2233 (1997)). Bone marrow cells may be obtained from ilium, sternum, tibiae, femora, spine and other bone cavities.

The manner in which target cells may be separated from other cells is not critical to this invention. Various procedures may be employed and include physical separation, magnetic separation using antibody-coated magnetic beads, affinity chromatography, and cytotoxic agents joined to a monoclonal antibody or used in conjunction with a monoclonal antibody. Also included is the use of fluorescence activated cell sorters (FACS) wherein the cells can be separated on the basis of the level of staining of the particular antigens. These techniques are well known to those skilled in the art and are described in various references including U.S. Pat. Nos. 5,061,620; 5,409,8213; 5,677,136; and 5,750,397; and Yau et al., Exp. Hematol. 18:219-222 (1990).

The order of cell separation is not critical to the invention, and specific cell types may be separated either prior to genetic modification with a MuSK-R or mMuSK-R or after genetic modification. Preferably cells are initially separated by a coarse separation followed by using positive and/or negative selection. In humans the surface antigen expression profile of an enriched hematopoietic stem cell population may be identified by CD34⁺Thy-1⁺Lin⁻. Other nonlimiting enriched phenotypes may include: CD2⁻, CD3⁻, CD4⁻, CD8⁻, CD10⁻, CD14⁻, CD15⁻, CD19⁻, CD20⁻, CD33⁻, CD34⁻, CD38^{lo/-}, CD45RA⁻, CD59^{+/-}, CD71⁻, CDW109⁺, glycophorin⁻, AC133⁺, HLA-DR^{+/-}, and EM⁺. Lin⁻ refers to a cell population selected on the basis of lack of expression of at least one lineage specific marker, such as, CD2, CD3, CD14, CD15 and CD56. The combination of expression markers used to isolate and define an enriched HSC population may vary depending on various factors and may vary as other express markers become available. Murine HSCs may be identified preferably by kit⁺Thy-1.1^{lo}Lin^{-/lo}Sca-1⁺ (KTLS). Other phenotypes are well known. (U.S. Pat. No. 5,061,620).

It has been shown that CD3 is expressed on most T cells, and that these cells express on the cell surface CD2, CD4, CD5, and CD8 antigens. Other well known useful T cell markers include CD54RA and T cell antigen receptor (TCR), α, β-TCR and γ, δ-TCR. B cells may be selected, for example, by expression of CD19 and CD20. Myeloid cells may be selected for example, by expression of CD14, CD15 and CD16. NK cells may be selected based on expression of CD56 and CD16. Erythrocytes may be identified by expression of glycophorin A. Neuronal cells may be identified by NCAM and LNGFR (Baldwin et al., J. Cell Biochem., 15:502 (1996)). Vascular endothelial cells may be identified by VEGFR2, CD34, P-Selectin, VCAM-1, ELAM-1 and ICAM-1 (Horvathova et al., Biol. Trace Elem. Res., 69:15-26 (1999). One skilled in the art is aware of other useful markers for identification of various cell types.

Once a population containing the target cells is harvested. The target cells are cultured in a suitable medium comprising a combination of growth factors that are sufficient to maintain growth. Methods for culturing target cells are well known to those skilled in the art., and reference is made to Freshney, R. I. “Culture of Animal Cells, A Manual of Basic Techniques”, Wiley-Liss, Inc (1994). Various culture media are commercially available and non -limiting examples include DMEM, IMDM, X-vivo 15 and RPMI-1640. The formulations may be supplemented with a variety of different nutrients and growth factors. Non-limiting examples of supplemental compounds which may be used are TPO, FL, KL, IL-1, IL-2, IL-3, IL-6, IL-12, IL-11, stem cell factor, G-CSF, GM-CSF, Stl factor, MCGF, LIF MIP-1α and EPO. These compounds may be used alone or in any combination, and preferred concentration ranges may be readily determined from the published art.

The medium can be serum free or supplemented with suitable amounts of serum such as fetal calf serum, autologous serum or plasma. If cells or cellular products are to be used in humans, the medium will preferably be serum free or supplemented with autologous serum or plasma (Lansdorp et al., J. Exp. Med. 175:1501 (1992) and Petzer et al. PNAS 93:1470 (1996). When murine stem cells are cultured, a preferred non-limiting medium includes mIL-3, mIL-6 and mSCF. Other molecules can be added to the culture media, for instance, adhesion molecules, such as fibronection or RetroNectin™ (Takara Shuzo Co., Otsu Shigi, Japan).

The seeding level is not critical and will depend on the type of cells used, but in general the seeding level will be at least 10 cells per ml, more usually at least about 100 cells per ml and generally not more than 10⁶ cells per ml when the cells express CD34.

In vitro systems for measurement of mammalian stem cell activity include the long-term culture initiating cell assay (LTCIC) and the cobblestone-area-forming cell (CAFC) assay. (Pettengell et al., Blood 84:3653 (1994); Breems et al., Leukemia 8:1095 (1994); Reading, et al., Exp. Hem. 22:786 (Abst # 406) (1994); and Ploemacher et al., Blood 74:2755 (1989)). In the CAFC assay a sparsely plated cell population is simply tested for its ability to form distinct clonal outgrowths (or cobblestone areas) on a stromal cell monolayer over a period of time. This assay gives frequency readouts that correlate with LTCIC and are predictive of engraftment in in vivo assays and patients. A particularly preferred CAFC assay is described in Young et al., Blood 88:1619 (1996). Flow cytometry can be used to subset hematopoietic cells from various tissue sources by the surface antigens they express. A combination of these assays may be used to test for target cells that are genetically modified according to the invention.

In one preferred embodiment the invention concerns a method of identifying genetically modified mammalian cells, particularly human cells comprising introducing a polynucleotide sequence encoding a MuSK-R or mMuSK-R as a selectable marker operatively linked to a promoter into the target cell to form a genetically modified cell; allowing expression of the MuSK-R or mMuSK-R in the genetically modified cell; and identifying said genetically modified cell expressing the MuSK-R or mMuSK-R.

In a most preferred embodiment the polynucleotide sequence encodes mMuSK-RI, mMuSK-RII or a mMuSK-R derived from the MuSK-R set forth in SEQ ID NO. 1 or a sequence substantially similar to said sequence with minor changes. A polynucleotide is said to “encode” a polypeptide if, in its native state or when manipulated by methods well known to those of skill in the art it can be transcribed and/or translated to reproduce a polypeptide or fragment thereof. A construct or vector including the MuSK-R or mutant thereof may be incorporated into the target population by any means of genetic transfer or modification known in the art.

The term “genetic modification” refers to any addition, deletion or disruption to a cells normal nucleotides and the methods of genetic modification are intended to encompass any genetic modification method of exogenous or foreign gene transfer or nucleic acid transfer into mammalian cells (particularly human hematopoietic cells). The term includes but is not limited to transduction (viral mediated transfer of host DNA from a host or donor to a recipient, either in vivo or ex vivo) and transfection (transformation of cells with isolated DNA genomes), including liposome medicated transfer, electroporation, calcium phosphate coprecipitation and others. Reference is made to Kriegler, M. Gene Transfer & Expression a Laboratory Manual, W. H. Freman & Company NY (1990)). Methods of transduction include direct co-culture of cells with producer cells (Bregni et al., Blood 80:1418-1422 (1992)) or culturing with viral supernatant alone with or without appropriate growth factors and polycations (Xu et al., Exp. Hemat. 22:223-230 (1994)).

In a preferred embodiment the target cells are transduced with a retroviral vector as previously described. The host cell range that may be infected is determined by the viral envelope protein. The recombinant virus can be used to infect virtually any other cell type recognized by the env protein provided by the packaging cell, resulting in the integration of the viral genome in the transduced cell and the stable incorporation of the foreign gene product. In general, murine ecotropic env of MoMLV allows infection of rodents' cells, whereas amphotropic env allows infection of rodent, avian and some primate cells including human cells. Recently, the G-glycoprotein from vesicular stomatitis virus (VSV-G) has been substituted for the MoMLV env protein. (Burns et al., Proc. Natl. Acad. Sci. USA 90:8033-8037 (1993); and WO92/14829). Xenotropic vector systems also exist which allow infection of human cells.

Once the target cells are genetically transformed by introduction of a MuSK-R or mMuSK-R nucleic acid sequence as the selectable marker, and optionally with a second nucleic acid sequence encoding a protein of interest, the modified cells expressing the MuSK-R or mMuSK-R may be identified by numerous techniques known in the art. The term “identify” or “identification” used herein in reference to genetically modified cells, unless indicated otherwise, means to mark, to purify, to enrich, to select, to isolate or to separate. Identification may be by a single or multiple steps. In one embodiment, the identified genetically modified cells are identified and separated in the same step.

Methods of identifying the target cells expressing MuSK-R or mMuSK-Rs include well known techniques such as antibody selection, particularly immunoselection; nucleotide selection by northern blots or by southern blots; PCR amplification of genomic DNA; protein detection by western blots; reverse transcription of mRNA and amplification with PCR; and FISH wherein chromosomes are analyzed by fluorescence in situ hybridization with a liquid phase DNA (Lawrence et al., Science, 249: 928-932 (1990)).

In a preferred embodiment, the method of identifying mammalian cells includes exposing the target cells to an antibody wherein the antibody specifically recognizes and binds to the cells expressing the mMuSK-R and does not bind to the cells lacking expression of mMuSK-R. The bound cells are then separated from cells that do not bind to the antibody.

Antibodies may be obtained by methods well known in the art and reference is made to Harlow et. Al., “Antibodies: A Laboratory Manual: (1988), Biosupplynet Source Book (1999) Cold Spring Harbor Laboratory Press. Polyclonal antibodies that are reactive to the antigen of interest may be used or monoclonal antibody producing cell clones may be generated. According to the invention, the antibody must recognize the extracellular domain of the MuSK-R or mMuSK-R selectable marker. More particularly if parts of the extracellular domain are modified, for example by deletion, the antibody should recognize an epitope of the remaining amino acid sequence of a mMuSK-R.

Particularly preferred antibodies are monoclonal antibodies that specifically recognize and bind to a mMuSK-R derived from or substantial similar to the MuSK-R sequence as set forth in SEQ ID NO:2. These antibodies are referred to as “α-MuSK-R” and the term encompasses any antibody or fragment thereof, either native or recombinant, synthetic or naturally derived which retains significant specificity to bind to a mMuSK-R derived from or substantially similar to the sequences set forth in SEQ ID NO:1 and 2. Exemplary of a α-MuSKR are the monoclonal antibodies referred to as H1, H2 and H4 described in the Example section G and produced by the deposited hybridomas.

Hyridomas producing antibodies to mMuSK-RI and mMuSK-RII designated H1, H2 and H4 have been deposited with the American Type Culture Collection (ATCC) 10801 University Blvd., Manassas, Va. 20110 on Mar. 22, 2000 and have been given ATCC Accession Nos. PTA-1547, PTA-1548, and PTA-1549, respectively.

The H1 monoclonal antibody is most preferred for identifying and further selecting target cells expressing the selective markers. Additionally, an antibody may be used in the methods according to the invention wherein the antibody binds specifically to an epitope in the extracellular domain as recognized by the antibody H1.

The α-MuSKR may be identified and assayed in vitro by a range of methods known in the art including gel diffusion, immunoassay, immunoelectrophoresis and immunofluorescence. Once the target cells are labeled they can be incubated with the α-MuSKR.

A secondary antibody may also be used to further identify or select antibody coated cells, if the secondary antibody is coupled to either a fluorophore or immuno-magnetic beads. The genetically modified cells expressing the selectable marker may then be selected by flow cytometry including FACS or by using a magnet to select bead-coated cells (U.S. Pat. No. 5,011,912). In brief, a primary α-MuSKR can be conjugated to a fluorophore, such as fluorescein isothiocyanate (FITC), phycoerythrin (PE), cy-chrome (CyC), allophycocyanine (APC), tricolor (TC) or Texas Red (TX). If the primary antibody is not conjugated to a fluorophore, a secondary antibody that is conjugated to a fluorophore may be introduced into the cell sample containing the cells that express mMuSK-R and which is recognized by the primary antibody. The primary antibody is attached to the mMuSK-R. Separation may be achieved by the fluorescence activated cell sorter (FACS).

FACS can also be used to separate cells expressing a tag sequence. A tag is a small amino acid sequence of approximately 10-20 amino acid which can be recognized by an antibody. Non-limiting examples of tags include, HA (hemagglutinin), myc tag, his tag, and FLAG® (Kunz et al., J. Biol. Chem 267: 91091 (1992)) which may be bound to a primary antibody specific to the tag. Tag products are available commercially. For example, from Eastman Kodak Company, New York. In one embodiment, the target cells will be genetically modified with a construct including the mMuSK-R and a polynucleotide sequence encoding a tag polypeptide. The modified cell will express the tagged selective marker at the cell surface. Anti-tag monoclonal antibodies, can be used to identify the cells expressing tagged MuSK-R at the cell surface. Anti-FLAG® is described in U.S. Pat. No. 5,011,912. Reference is also made to U.S. Pat. Nos. 4,703,004, 4,782,137 and 4,851,341 and Brizzard et al., Biotechniques 16:730 (1994).

The genetically modified cells identified according to the methods of the invention may be expanded, either prior to or after identification or selection by culturing the cells for days or weeks in appropriate culture media, with or without supplements by means well known in the art.

The genetically modified cells identified according to the invention may further be used in an autologous or allogeneic setting wherein the modified target cells, preferably hematopoietic cells, most preferably stem cells or T-cells are expanded and then used in gene therapy for example in bone marrow transplantation, graft facilitation, or immune reconstitution. The expanded cells including the mMuSK-R may be infused into a subject. Samples may be taken and then retested for the MuSK-R or mMuSK-R selectable markers by FACS analysis, PCR or FISH as described above to determine the persistence of the genetically modified cells and further to assess efficiency of transformation, particularly efficiency of transduction.

The invention generally described above will be more readily understood by reference to the following examples, which are hereby included merely for the purpose of illustration of certain embodiments and are not intended to limit the invention in any way.

### Experimental

### EXAMPLE 1

A. Isolation of Human MuSK-R cDNAs:

MuSK-R is isolated by PCR from fetal skeletal muscle cDNA (Marathon cDNA, Invitrogen) using primers flanking the 5′ and 3′ of the MuSK-R cDNA.

The following primers obtained from Operon Technologies, Inc. are used to amplify MuSK-R cDNA:

| | | |
|---|---|---|
| MuSK21FN: | CGT CCT GCG TGA GCC TGG ATT AAT C | SEQ ID NO: 3 |
| MuSK34FN: | GCC TGG ATT AAT CAT GAG AGA GCT C | SEQ ID NO: 4 |
| MuSK2666RN: | CGA GGC CTG TCT TCA ACC TTA GAC ACT CAC AGT TCC | SEQ ID NO: 5 |
| | CTC TGC | |

The 5′ primer MuSK21FN covers 25nucleotide (nt) before the start codon, the second 5′ primer MuSK34FN covers the start codon (aa 1) of MuSK-R and surrounding sequence. The 3′-primer MuSK2666RN covers the stop codon of MuSK-R and surrounding sequence. Using primers MuSK2FN, MuSK34FN and MuSK2666RN results in the amplification of a DNA fragment of ˜2600 bp that encodes a MuSK-R wt protein.

The following PCR reaction is performed: Marathon cDNA (˜2 ng) is mixed with Advantage cDNA buffer (10 mM Tris-HCl (pH=7.5 at 42° C.), 50 mM KCl, 2.5 mM MgCl₂, 0.001% Gelatin), 2.5 μmol dATP, 2.5 μmol dCTP, 2.5 μmol dGTP, 2.5 μmol TTP), 1 μg primer MuSK21FN, 1 μg primer MuSK2666RN, 1 μl Advantage cDNA polymerase, and water in a final volume of 50 μl. The PCR is performed as follows: Cycle 1: 94° C. for 5 min, Cycle 2-11: 94° C. for 0.5 min, 63° C. for 1 min, 68° C. for 6 min, and Cycle 12: 68° C. for 10 min.

The reaction is cooled to 4° C. in the PCR machine, and the amplified cDNA is ethanol precipitated with 0.3 M sodium acetate. The pellet is washed once with 70% ethanol, dried and resuspended in 100 μl H₂O.

10 μl of the above PCR reaction is then reamplified. The reaction mix contains for the second round of amplification step in addition to 10 μl of the above PCR reaction: Pfu buffer (20mM Tris-HCl (pH8.8), 2 mM MgSO₄, 10 mM KCl, 10 mM (NH₄)₂SO₄, 0.1% Triton X-100, 0.1 mg/ml BSA), 2.5 μmol of each dNTP (dATP, dCTP, dGTP, dTTP), 1 μg primer MuSK34FN, 1 μg primer MuSK2666RN, 5 U Pfu Turbo Polymerase (from Pyrococcus furiosus) and water in a final volume of 50 μl. The PCR is performed as follows: Cycle 13: 94° C. for 5 min, Cycle 14-43: 94° C. for 0.5 min, 62° C. for 1 min, 72° C. 6 min, and Cycle 44: 72° C. for 10 min.

The reaction is cooled to 4° C. in the PCR machine and the amplified cDNA is ethanol precipitated with 0.3 M sodium acetate. The pellet is washed once with 70% ethanol, dried and resuspended in 20 μl H₂O. The PCR reaction is loaded on a 1×TAE gel. A band with the size of ˜2600 bp is isolated from the gel and cloned into the SrfI restriction site of pPCR-Script Amp vector (Stratagene, Calif.) according to the manufacturer's protocol. The resulting vector is called pPCR-Script MuSK-R-wt. The correctness or the subcloned PCR product is confirmed by restriction analysis and sequencing by methods well known in the art. (The nucleotide sequence is illustrated in SEQ ID NO.1)

B. Generation of Mutations in the Intracellular Domain of MuSK-R by PCR:

The primers MuSK1380F, MuSK1657R, and 1747R are used to generate intracellular deletion mutants of MuSK-R from the plasmid pPCRScriptMuSK-R. The primer sequences are as follows wherein p means phosphorylated:

| | |
|---|---|
| Primer 1380F: 5′ pCG GCC TGT GCC AGA CTG CCA CAT CTA G | (SEQ ID NO: 6); |
| Primer 1657R: 5′ pCG TCT AGG TGA GGG TTA CTG CTG CTG ATT CTC | (SEQ ID NO: 7); and |
| Primer 1747R: 5′ pGG TTA ACC CTA TTC AAT GTT ATT CCT TGA ATA CTC CAG | (SEQ ID NO: 8). |

Using primer pair MuSK1380F and 1657R results in the deletion of amino acid residues 538-879 of MuSK-R, using primer pair MuSK138OF and 1747R results in the deletion of amino acid residues 577-879. The two mutant forms of MuSK-R are designated MuSK-RA538-879 (MuSK-RI) and MuSK-RA577-879 (MuSK-RII). In both MuSK-RI and MuSK-RII most of the intracellular domain of MuSK-R as shown in FIG. 2 is deleted. While not meant to limit the invention in any manner, it is believed that both truncations result in a deletion of the kinase domain and most of the substrate binding motifs of the wt MuSK-R illustrated in FIG. 2.

The 5′ primer MuSK1380F covers the nucleotide sequence 1333-1410 of the MuSK-R. The 3′-primers MuSK1657R and 1747R contain stop codons in place of amino acid 538 and 577 of MuSK-R. Using primer MuSK138OF with MuSK1657R or MuSK1747R results in the amplification of MuSK-R nucleotide sequence 1333 to 1614 that has a stop codon in the position of amino acid 538 or nucleotide sequence 1333-1728 that has a stop codon in the position of amino acid 577, respectively. The PCR reaction includes ˜10 ng hMuSK-R wt DNA, 1×Pfu buffer, 1 μg of primer MuSK1380F and either 1 μg primer MuSK1657R or MuSK1747R, 2.5 μmol of each dNTP, 5 U Pfu polymerase and H₂O in a final volume of 50 μl. The PCR reaction is performed as follows: Cycle 1: 95° C. for 5 min, Cycle 2-31: 95° C. 0.5 min, 60° C. for 1 min, 72° C. for 4 min, Cycle 32: 72° C. for 10 min. The PCR reaction is cooled to 4° C. in the PCR machine and then loaded on a 1 × TAE gel.

The two PCR products MuSK-RI (nt 1380-1614) and MuSK-RII (nt 1380-1728) are cloned into the SrfI site of pPCR-ScriptAmp (Stratagene) according to the manufacturer's protocol. As the 5′ coding sequence from MuSK (nt 1-1379) is missing in these constructs, this sequence is excised from the plasmid pPCR-Script MuSK-wt using restriction sites NaeI and AatII. The two pPCR-Script vectors containing the modified MuSK sequence nt 1-1614 and 1-1726 are called pPCR-Script-MuSK-RI and pPCR-Script MuSK-RII, respectively. The correctness of the vectors are confirmed by restriction analysis and sequencing by methods well known in the art.

C. Generation of Retroviral Vectors Containing Mutated MuSK-Rs and Viral Supernatants:

Wild-type and mutant MuSK-R are excised from pPCRScriptMuSK-Rwt, pPCRScriptMuSK-RI and pPCRScriptMuSK-RII using the NotI and XhoI site and are cloned into the multiple cloning site of the Moloney Murine Leukemia Virus (MOMLV) based retroviral vector pG1a (GTI, Maryland) which is cut with NotI and XhoI. The retroviral vectors are designated pG1aMuSK-R, pG1aMuSK-RI and pG1aMuSK-RII. The constructs pG1aMuSK-R, pG1aMuSK-RI and pG1aMuSK-RII are cotransfected into human embryonic kidney cells 293T (293T cells) (Gary Nolan, Stanford) with an envelope construct pCiGL that permits expression of the Vesicular Stomatitis Virus G-Protein (VSV-G envelope) under the control of the cytomegalovirus (CMV) promoter. Also cotransfected into 293T cells is the packaging construct pCiGP (encoding MoMLV gag-pol under the control of the CMV promoter) using the CaCl₂ technique (Clontech). Reference is made to WO 97/21825 and Rigg et al. Virology 218: 290-295 (1996).

Viral supernatants are collected 24, 48, and 72 hours after transfection. Supernatants are centrifuged at 1200 rpm in a Beckman GS-6KR centrifuge to remove particulate material, and either used immediately to transduce cells or frozen in a dry ice/methanol bath. The viral supernatants are used to transduce the packaging cell line ProPak-A-6 (PPA-6) (Systemix, Inc.). The PPA-6 cell line is a derivative of 293T cells expressing the MLV amphotropic envelope and MLV gag/pol stabley under the control of the CMV promoter (Rigg et al. supra). The positively transduced PPA-6 cells are sorted by bead selection (described in section F). Supernatants from PPA-6 cells are collected on day 2, 3 and 4 after transduction and treated as described for 293T cells. The so generated supernatants of PPA-6 cells contain recombinant viral particles that have the amphotropic envelope and are used to transduce human primary cells and cell lines as described below.

D. Tissue Culture and Cell Lines:

The following cell lines and primary cells are used: (a) human T cell line, CEMSS (Frederico et al., J.Biol. Regul. Homeost. Agents, 7: 41-49 (1993)) (b) human embryonic kidney cells 293T (293T) (Pear et al., Proc. Natl. Acad. Sci. USA 90:8392-8396 (1993)), and (c) PPA-6 (Rigg et al., supra). A CEMSSMuSK-R cell line is generated by transducing CEMSS cells with PPA-6 supernatants that are made using the pG1a-MuSK-Rwt construct.

Cells are cultured in a Steri-Cult 200 incubator (Forma-Scientific) at 5% CO₂. Media (DMEM, Iscove's medium, RPMI), PBS, and sodium pyruvate are obtained from JRH Biosciences (CA), FBS from Hyclone (UT), L-glutamine, Trypsin from Life Technologies (MD), ITS (insulin/transferrin/sodium selenite), PHA (phythemaglutinin), Interleukin-2 (Il-2) from Sigma (Missouri).

293T cells and PPA-6 cells are cultured in DMEM, 10% FBS, 1% sodium pyruvate, and 1% L-glutamine. CEMSS cells are cultured in RPMI, 10%FBS, 1% L-glutamine, and 1% sodium pyruvate. Hybridoma cells are grown in (hypoxanthine aminopterin thymidine (HAT) media or HT media (Iscove's medium, 10% FBS, 5% hybridoma cloning factor (Igen; MD) plus 0.5 mM hypoxanthine, 4 μM aminopterin, 16 μM thymidine in HAT medium or 0.5 mM hypoxanthine, 16 μM thymidine in HT medium).

In order to passage adherent cells (293T and PPA-6) cells are washed once with PBS, then trypsinized for 5 min and subsequently split into new tissue culture flasks (VWR; NJ).

E. Transduction of PPA-6 and Human T Cell Line:

10⁶ cells/ml from step (D) are transduced with 1-3 ml of viral supernatant, that had been either generated from 293 T cells or PPA-6 cells, by spinoculation with 8 μg/ml protamine sulfate (Sigma, Missouri). Using standard techniques, spinoculation is done at 37° C. for 3 hrs at 2750 rpm for PPA-6 and CEMSS cells. PPA-6 cells are transduced in 6 well plates, and CEMSS cells in 6-ml tubes.(VWR)

F. FACS Analysis and Immuno-magnetic Bead Selection of Cells that Express MUSK-RI and MUSK-RII:

FACS analysis is done on a FACScan (Becton Dickinson). The following antibodies and reagents are used for staining. CD4-FITC (Caltag), propidium iodide (PI), goat anti-mouse IgG-PE (Caltag), goat anti-mouse IgG coupled magnetic beads (Dynal, Oslo), anti-MuSK-R polyclonal serum, and anti-MuSK-R hybridoma supernatant (see section G). All antibodies are titrated and optimal concentrations are used. 1×10⁶ cells are stained in 50 μl of PBS/2% FBS for 20 to 60 minutes at 4° C. When a secondary antibody is used, the cells are washed once with 2 ml of PBS/2%FBS, then again incubated in 50 μl PBS/2%FBS and the secondary antibody is added. Before the FACSanalysis the cells are again washed once with PBS/2%FBS, centrifuged and resuspended in 500 μl PBS/2%FCS containing 1 μg/ml PI. FACSanalysis is performed on a FACSscan (Becton-Dickinson Immunocytometry Group, CA) according to manufacturer's instructions.

In order to isolate cells by bead selection, the cells are stained with an anti-MUSK-R antibody. For this purpose the 10⁷ cells/ml are incubated with 1-3 ml anti-MuSK-R hybridoma supernatant in PBS/2%FBS for 1 hr on ice with occasional shaking. The cells are washed 3 times with PBS/2%FCS and then anti-IgG antibody coupled magnetic beads, that can recognize anti-MuSK-R antibodies, are added (˜5 beads per positive cell). The cells are incubated for 1 hr on ice. Cells that express MuSK-R are selected by positive selection with a Dynal magnet (Dynal, Oslo) for 10 min. The unbound cells are removed and the MuSK-R expressing cells are put into culture as described in section D.

G. Generation of a Monoclonal Antibody Against the Extracellular Domain of MuSK-R:

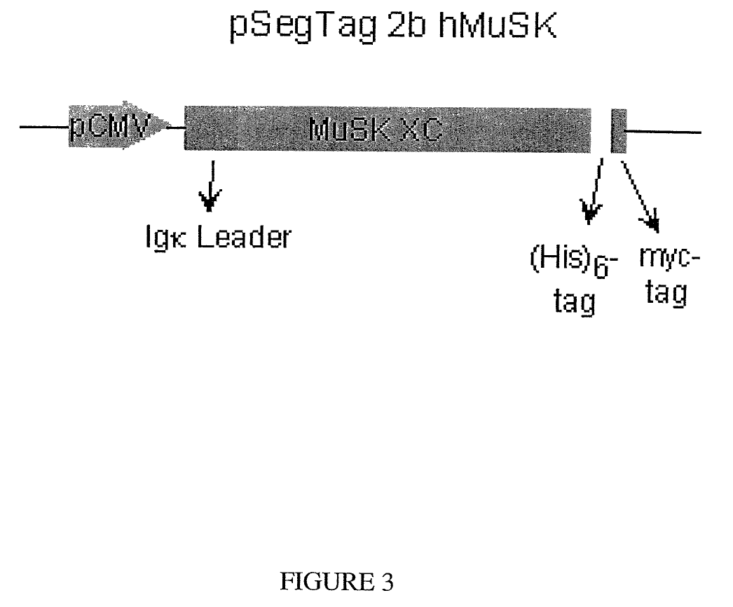

To generate monoclonal antibodies against the extracellular domain (XC) of MuSK-R, MuSK-R XC is amplified by PCR and cloned into the expression construct pSecTag2b (Invitrogen). Cloning the XC domain of MuSK-R into the multiple cloning site (MCS) of the plasmid pSecTag2b allows for the expression of the XC under the control of the CMV promoter. In addition, the plasmid contains the sequence of a myc and (His)₆-tag after the multiple cloning site, which allows to fuse the protein of interest (MuSK-RXC) to the myc and (His)₆-tag. The signal peptide of MuSK-R is replaced by the IgK leader. (FIG. 3). The extracellular domain of MuSK-R without the signal peptide is amplified by PCR using the following primers wherein p means phosphorylated:

| | |
|---|---|
| MuSK 116FPC: 5′ pCT TCC AAA AGC TCC TGT CAT CAC C | SEQ ID NO: 9 and |
| MuSK 1532RC: 5′ pCC AGT CAT GGA GTA TGT AGG TGA GAC | SEQ ID NO: 10 |

Primer MuSK116FPC starts with the sequence after the signal peptide (nt 69-93). Primer MuSK1532RC covers the sequence before the transmembrane domain starts and the first 2 amino acids of the transmembrane domain corresponding to nucleotide sequence 1462-1586 of SEQ I NO:1. For the PCR reaction ˜10 ng hMuSK-R wt DNA are mixed with 1 × Pfu buffer, 1 μg of primer MuSK116FPC and 1 μg primer MuSK1532RC, 2.5 μmol of each dNTP, 5 U Pfu polymerase and H₂O in a final volume of 50 μl. The PCR reaction is performed as follows: Cycle 1: 95° C. for 5 min, Cycle 2-7: 96° C. for 0.5 min, 60° C. for 1 min, 72° C. for 6 min, and Cycle 8-27: 95° C. for 0.58 min, 58° C. for 1 min, 72° C. for 6 min; Cycle 28: 72° C. for 10 min. The PCR reaction is cooled to 4° C. in the PCR machine and then gel-purified. The PCR fragment is cloned into to the EcoRV restriction site of pSecTag2b. In this way MuSK-R XC is cloned in frame with the Igk leader at the N-terminus and the myc- and (His)₆-tag at the C-terminus. The resulting plasmid is called pSecTag-hMuSK-R.

To express the MuSK-R XC the plasmid pSecTag-hMuSK-R is transfected into 293T cells by the CaCl₂ technique (as described in section C). 24 hrs after the transfection, the media is replaced with either fresh DMEM/10%FBS or serum-free X-Vivo 15. Supernatants of the transfected cells are collected after 48 and 72 hrs. A total of 400 ml supernatants are collected and are frozen at −80° C. until the supernatants are purified.

The MuSK-R XC is purified from tissue culture supernatants by immobilized metal affinity chromatography. The metal ion is 0.1 M NiCl₂. The column is a 1 or 5 ml Pharmacia metal HiTrap chelating sepharose column. The equilibration buffer (Buffer A) consisted of 20 mM Na₂HPO₄ pH 7.4, 1M guanidine hydrochloride, 1M NaCl, filtered through 0.2 μM cellulose acetate filter. The elution buffer (Buffer B) is 20 mM Na₂HPO₄, pH 7.4, 1 M guanidine hydrochloride, 1M NaCl, 0.5 M imidazole, filtered through 0.2 μM cellulose acetate filter. The Pharmacia FPLC chromatography system is used to run columns, with FPLC director program software and a Pharmacia P50 pump. The purification is performed at 4° C.

The pump is primed with buffer A before the load is started. Before the column the column is attached, the load is pumped through until the pink color of the tissue culture media is seen at the connection so that the column is not washed with non-equilibration conditions.

The tissue culture supernatants are adjusted to contain 0.85 M NaCl, 1M guanidinium chloride and 40 mM imidazole and the pH is adjusted to 7.4. The column is equilibrated with 8% buffer B. The sample is loaded and the column then washed in above conditions for seven column volumes. MuSK-R is eluted at 30% Buffer B (150 mM imidazole) over eight column volumes. Fractions are collected from start of the run.

Each fraction is tested in a Dot Blot Assay (see below). Selected positive fractions are tested in Western Blot assays and Elisa (see below). Positive fractions are pooled and dialyzed in 10,000 MWCO membrane (Pierce Snakeskin) against PBS. After dialysis, the optical density is determined at OD₂₈₀. The samples are filtered through 0.2 μM filters and then concentrated in Centricon Centriprep 30 devices in a refrigerated Sorvall RT6000D according to manufacturer's protocol.

To test for positive samples in the dot blot assay, 10 μl of each fraction is pipetted on nitrocellulose. The membrane is dried, blocked with superblock and then probed for MuSK-R protein and developed as described for the India Western (see below).

Western Blots are performed by methods well known in the art. The materials (sample buffer, running buffer and gels) are obtained from Novex.

For western blot analysis 25 to 35 μl/fraction are used. Guanidine containing fractions are precipitated in ice-cold ethanol and are stored on ice for 15 minutes or overnight at 4° C. The samples are centrifuged in refrigerated microcentrifuge (TOMY) at 14,000 rpm for 10 min. The supernatant is discarded, ice-cold acetone is added and centrifuged as before. The pellet is resuspended in SDS sample buffer (Novex) with 5% β-mercaptoethanol in a final volume of 50-70 μl. The samples are denatured at >90° C. for 5 minutes, briefly centrifuged, and 25-35 μl loaded on a 4-20% gradient gel. The gel is blotted onto 0.45 μM nitrocellulose for 1.4 hours at 100 volts, using the Biorad wet transfer blotting cassette with tris-glycine-methanol transfer buffer (25 mM trizma Base, 192 mM glycine, 20% methanol). After blotting the gel, the blot is blocked in Pierce TBS superblock for 10 minutes with mild agitation. The blot is washed twice in TBST (50 mM Tris, pH 7.5, 150 mM NaCl, 0.05% Tween 20) for five min per wash on a rotating platform. The Pierce India™ His-HRP Probe is diluted to 1:5000 in TBST and the blot is incubated with the probe for 1 hour at room temperature and washed 3 times in TBST. After that, horseradish peroxidase reagent (Sigma Fast HRP Insoluble Substrate D4418) is added to the blot and the blot is developed. The blot is washed in three changes of water to stop development. Alternatively an mouse anti-c-myc antibody, (Santa Cruz Biotechnology; CA) is used to detect recombinant MuSK-R protein. This antibody is diluted in superblock to 1 μg/ml final concentration. The blot is washed three times with TBST and then a goat anti-mouse IgG-HRP antibody (Sigma) is added at 1:5000 dilution in superblock. The blot is incubated for 1 hour at room temperature, with gentle agitation and developed as described above with Fast HRP insoluble substrate (Sigma). The recognized protein traveled at about 85 kD on the SDS PAGE, and it is considered to be 19 kD heavier due to glycosylation.

The recombinant MuSK-R protein is injected into 3 different Balb/c mice. For this purpose 25-50 μg are mixed with 2.25 mg alhydrogel and 100 μg MDP (muranyl dipeptide; Pierce) in a final volume of 200 μl and injected 5 times every 14 days subcutaneously. After the 3^{RD} and the 5^{TH} injection serum of the 3 mice are tested for reactivity against MuSK-R by FACSanalysis and Elisa. For FACSanalysis the 5×10⁵ cells of cell lines CEMSS and CEMSSMuSK-R are used. Both the presera and sera are diluted 1:100. 1:300, 1:900 and 1:2700. A rat anti-mouse IgG-PE antibody is used as a secondary reagent at a 1:20 dilution. For the Elisa, 96 well plates are coated with 50 μl of 10 μg/ml anti-mouse IgGF_{c} (Jackson; Maine). The plates are incubated with various dilutions of sera (1:100 to 1:218700), subsequently with MuSK-R protein and with Nickel activated horse radish peroxidase at a 1:1000 dilution (HRP, Pierce). Nickel activated HRP is binding to the recombinant MuSK-R protein via the (His)₆ tag. To develop the Elisa, the plate is incubated with TMP peroxidase substrate (Zymed; CA). In both assays one mouse shows the highest reactivity against native and recombinant MuSK-R. This mouse is boosted with a 6^{th} injection of 200 μg MuSK-R protein in PBS. The injection is done subcutaneously and intravenously. 1 week later the spleen is removed, lymphocytes isolated with lympholite M (Accurate Chemicals) and fused, using 50% polyethylene glycol to the myeloma cell line P3X63AG8.0653 using standard procedures. The resulting hybridomas are grown in bulk in HAT media for one week. Viable cells are recovered using lympholite M and cultured in HAT media plus cloning factor (Igen). After the hybridoma are grown for another week, a batch of the cells are cryopreserved in HAT media plus 10% DMSO. Another batch of the cells is subdivided into individual clones by FACSsorting using the single cell deposit unit. The cells are sorted by forward and side scatter and for PI negative cells. The cells are grown up in HT media for two weeks. The supernatants are tested by Elisa and FACS (as described above) for monoclonal antibodies that can recognize native and recombinant MuSK-R protein. The antibodies are isotyped in an Elisa assay by using secondary antibodies that react with IgG1, 2a, 2b, 3, IgM, κ, and λ (Caltag, Calif.).

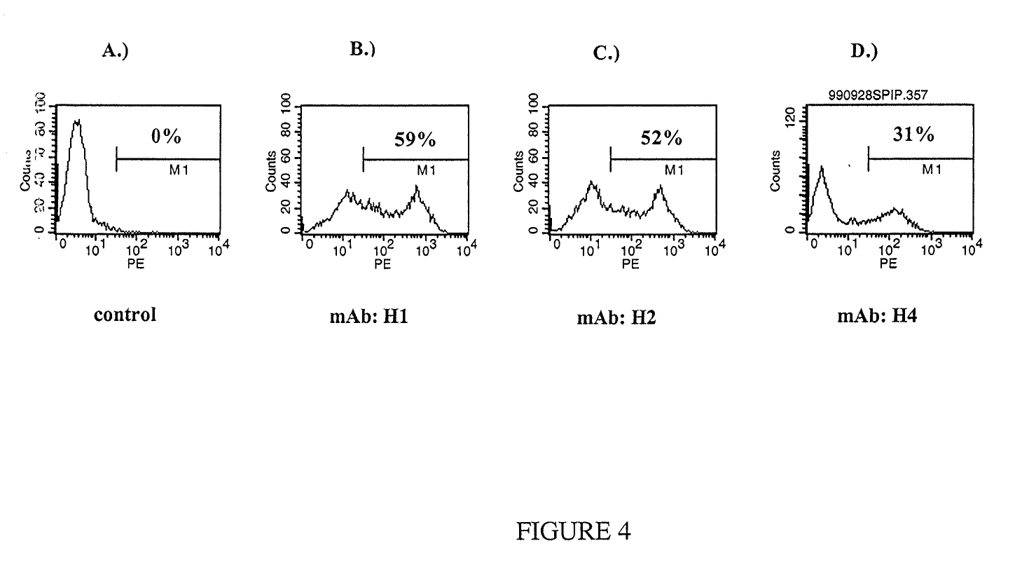

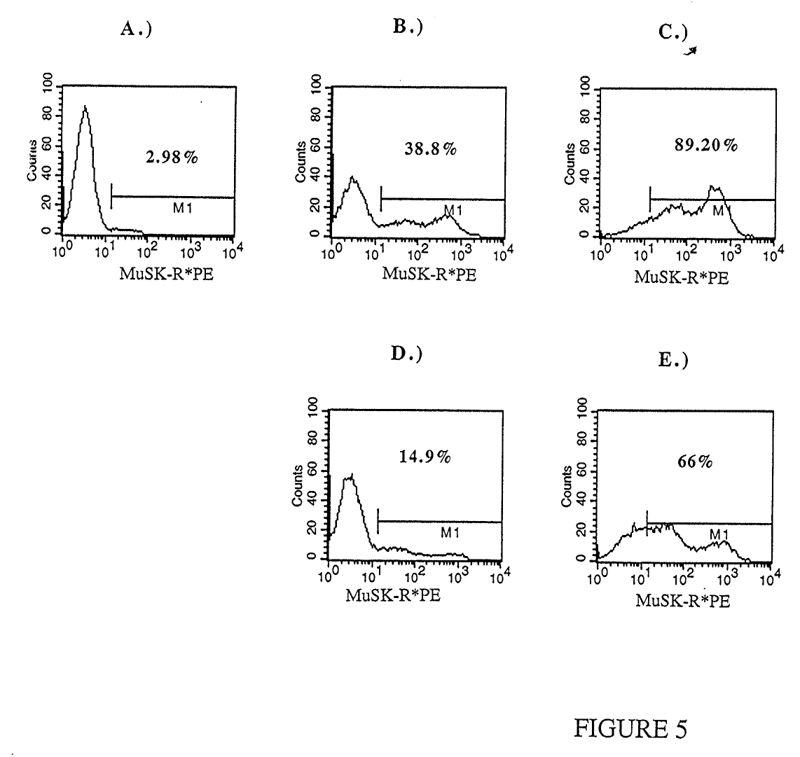

Three monoclonal antibodies are identified H1, H2 and H4. All three can react with MuSK-R expressed on the cell ine CEMSS-MuSK-R in a FACS assay. H1 is an IgG1, κ; H2 is IgG1, κ; H4 is IgM antibody. FIG. 4 shows expression of hMuSK-R on CEMSS cells and CEMSS-MuSK-R cells using the antibodies H1, H2 and H$. To detect the antibodies H1, H2 and H4 a secondary PE coupled to anti-mouse IgG is used. FIG. 5 illustrates expression of MuSK- on nontransduced CEMSS cells (panel A) and on CEMSS cells that are transduced with PP6-A supernatants so they express hMuSK-R (panel B) or mMuSK-RII (panel D). Both populations were enriched after immuno-magnetic bead selection as illustrated for hMuSK-R (panel C) and mMuSK-RII (panel E). The results of the experiments illustrated in FIG. 5 are performed using the monoclonal antibody H2.

It is claimed:

1. A method of identifying genetically modified mammalian cells comprising the steps of:
a) introducing a nucleic acid sequence encoding a mutated muscle specific tyrosine kinase receptor (mMuSK-R) operatively linked to a promoter into a mammalian cell to form a genetically modified cell;
b) allowing expression of the mMuSK-R in the genetically modified cell; and
c) identifying the cells expressing the mMuSK-R.

2. The method according to claim 1 wherein the mMuSK-R is a mutated sequence of the sequence encoded by the nucleic acid molecule set forth in SEQ ID NO. 1.

3. The method according to claim 1, wherein the mMuSK-R is a sequence having at least 150 amino acids deleted from the intracellular domain of a MuSK-R.

4. The method according to claim 1, wherein the mMuSK-R is a MuSK-R sequence having the kinase catalytic site deleted.

5. The method according to claim 2, wherein the mMuSK-R is mMuSK-RI or mMuSK-RII.

6. The method according to claim 1, wherein the identifying step is accomplished by contacting the genetically modified cells with an antibody.

7. The method according to claim 1, wherein the nucleic acid sequence encoding the mMuSK-R is introduced into the mammalian cell by a vector.

8. The method according to claim 6, wherein the vector is a retroviral vector.

9. The method according to claim 1, wherein the mammalian cells are hematopoietic cells.

10. The method according to claim 1, wherein the nucleic acid encoding the mMuSK-R is introduced in combination with a second nucleic acid encoding sequence wherein the second sequence encodes a protein of interest.

11. The method according to claim 1, further comprising the step of separating the identified cells expressing the mMuSK-R.

12. The method according to claim 1, wherein the identifying step separates the genetically modified cells from the non-modified cells.

13. A vector comprising a nucleic acid sequence encoding a mutated muscle specific tyrosine kinase receptor (mMuSK) operatively linked to a promoter wherein the mMuSK-R is derived from the sequence set forth in SEQ ID NO. 1 or a sequence substantially identical to the sequence set forth in SEQ ID NO:1.

14. A method of identifying genetically modified human hematopoietic cells comprising the steps of:
a) introducing a nucleic acid sequence encoding a muscle specific tyrosine kinase receptor (MuSK-R) into a human hematopoietic cell;
b) allowing expression of the MuSK-R in said cells; and
c) identifying the genetically modified hematopoietic cells from the non-modified hematopoietic cells.

15. A method of identifying genetically modified human hematopoietic cells comprising the steps of:
a) incorporating a nucleic acid sequence encoding a mutated muscle specific tyrosine kinase receptor (mMuSK-R) into a population of human hematopoietic cells;
b) introducing a heterologous DNA sequence which encodes a protein of interest into the population of human hematopoietic cells;
c) allowing expression of the mMuSK-R in said cells; and
d) identifying the genetically modified cells expressing the mMuSK-R.

16. The method according to claim 14, wherein both the heterologous DNA sequence encoding the protein of interest and the nucleic acid sequence encoding the mMuSK-R are introduced into the cells on the same vector.

17. A method for the immunoselection of transduced mammalian cells comprising the steps of:
a) transducing cells with a nucleic acid sequence encoding a mutated muscle specific tyrosine kinase receptor (mMuSK-R);
b) incubating the cells with an antibody which recognizes and binds specifically to the mMuSK-R; and
c) identifying the bound transduced cells.

18. The method according to claim 16, wherein the cells are transduced by a retroviral vector derived from the group consisting of moloney murine leukemia virus (MoMLV), myeloproliferative sarcoma virus (MPSV), murine embryonic stem cell virus (MESV), murine stem cell virus (MSCV) and spleen focus forming virus (SFFV).

19. The method according to claim 16, further comprising separating the identified bound transduced cells from non-bound cells.

20. The method according to claim 16, further comprising expanding the bound transduced cells.

21. A method of identifying mammalian cells expressing a protein of interest, comprising
a) introducing into a population of mammalian cells a nucleic acid sequence encoding a mutated muscle specific tyrosine kinase molecule (mMuSK-R), wherein said mMuSK-R can not effect signal transduction;
b) introducing a nucleic acid sequence comprising a DNA sequence encoding a protein of interest into said population;
c) culturing the mammalian cells under conditions which favor growth and expansion of said cells; and
d) identifying cells which express mMuSK-R thereby obtaining cells which express the protein of interest.

22. An antibody which binds specifically to the epitope recognized by the antibody H1 produced by the hybridoma deposited with the ATCC under accession number PTA-1548.

23. A method of identifying mammalian cells comprising the steps of:
a) introducing a nucleic acid sequence encoding a mutated muscle specific tyrosine kinase receptor (mMuSK-R) operatively linked to a promoter into a mammalian cell to form a genetically modified cell;
b) allowing expression of the mMuSK-R;
c) exposing the cells to a monoclonal antibody wherein said antibody recognizes and binds to the cells expressing the mMuSK-R and does not bind to the cells lacking expression of mMuSK-R; and
d) separating the cells that bind to the monoclonal antibody from cells that do not bind to the antibody.

24. The method according to claim 23 wherein the antibody is selected from the group consisting of H1, H2, H4 and an antibody which binds specifically to an epitope in the extracerllular domain as recognized by the antibody H1.
